(19) United States
(12) Patent Application Publication
(10) Pub. No.: US 2024/0263198 A1
(43) Pub. Date: Aug. 8, 2024

(54) METHODS AND ORGANISMS FOR PRODUCING GLYCOLIC ACID

(71) Applicant: LYGOS, INC., Berkeley, CA (US)

(72) Inventors: Jeffrey Dietrich, Berkeley, CA (US); Andrew Conley, Berkeley, CA (US); Chang-Ting Chen, Berkeley, CA (US); Christine Carle, Berkeley, CA (US); Johan Van Walsem, Berkeley, CA (US)

(21) Appl. No.: 18/560,601

(22) PCT Filed: May 10, 2022
(86) PCT No.: PCT/US2022/028503

§ 371 Date: Nov. 13, 2023

Related U.S. Application Data
(60) Provisional application No. 63/187,590, filed on May 12, 2021.

(51) Int. Cl. C12P 7/42; C12N 9/04; C12N 15/52

(52) U.S. Cl. CPC C12P 7/42; C12N 9/0006; C12N 15/52; C12Y 101/01001

(57) ABSTRACT

Provided herein are methods, organisms, and tools for producing glycolic acid from ethylene glycol, and glycolic acid produced thereby.

### CROSS-REFERENCE TO RELATED APPLICATIONS

This application is a U.S. Nation Phase Application of International Application No. PCT/US2022/028503 filed May 10, 2022, which claims the benefit of U.S. Provisional Patent Application No. 63/187,590 filed May 12, 2021, the entire disclosures of which are expressly incorporated herein by reference.

### FIELD

This invention relates to microbial fermentation. In particular, the invention relates to methods, organisms, and tools for producing glycolic acid from ethylene glycol, and glycolic acid produced thereby.

### STATE OF THE ART

There is a need for producing glycolic acid, which is an important monomer for polyester polymers, especially for producing glycolic acid without using chemical reagents that may be environmentally harmful, and/or for producing glycolic acid from renewable feedstock of non-petrochemical origin.

### SUMMARY

In one aspect, provided herein is a method for producing glycolic acid or a glycolate salt comprising culturing an organism that is a wild-type Pichia kudriavzevii or a wild-type Corynebacterium glutamicum, in a fermentation broth, for a sufficient period of time to produce glycolic acid, wherein the fermentation broth comprises ethylene glycol.

In another aspect, provided herein is a recombinant host cell that comprises a glycolic acid biosynthetic pathway, wherein the glycolic acid biosynthetic pathway comprises heterologous nucleic acids encoding a glycolaldehyde-producing enzyme and a glycolate-producing enzyme; wherein the heterologous nucleic acids are expressed in sufficient amounts to produce glycolic acid or a salt thereof (i.e., a glycolate salt). In another embodiment, the recombinant host cell is a yeast cell or a bacterial cell. In another embodiment, the recombinant host cell belongs to the genus selected from a group comprising Pichia, Issatchenkia, Candida, Corynebacterium or Escherichia. In one embodiment, the recombinant host cell is Pichia kudriavzevii.

In another aspect, provided herein is a method for producing glycolic acid or a glycolate salt comprising culturing an organism that is a recombinant host cell of the present disclosure for a sufficient period of time to produce glycolic acid or a salt thereof, wherein the fermentation broth comprises ethylene glycol. Without being bound by theory, glycolic acid is produced from ethylene glycol as schematically shown below:

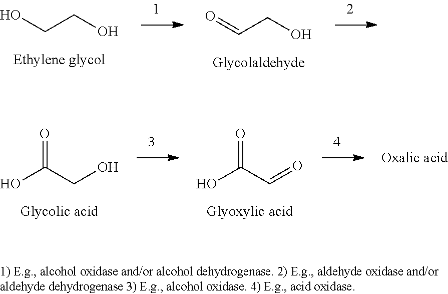

### Scheme 1

In another aspect, provided here is a method for producing a glycolic acid polymer that comprises culturing the wild-type or recombinant cell of this disclosure under fermentation conditions suitable to produce glycolic acid or a salt thereof. In some embodiments, the method comprises isolating the glycolic acid or salt thereof. In some embodiments, the method comprises optionally converting the glycolic acid or salt thereof to a glycolic acid derivative. A glycolic acid derivative includes without limitation a cyclic glycolide. In some embodiments, the method comprises producing a glycolic acid polymer using the isolated glycolic acid, the salt thereof, or the glycolic acid derivative.

In one embodiment, provided herein is a glycolic acid or a salt thereof containing carbon that has an isotopic distribution that is within measurement error of the isotopic distribution contained in terrestrial plant matter, indicating that the carbon was sourced from a sustainable source rather than petroleum. In some embodiments, non-petrochemical based component-containing compositions provided herein contain a ¹⁴C amount substantially higher than zero, such as about 1 parts per trillion or more. In one embodiment, the non-petrochemical based component-containing compositions comprise glycolic acid or a salt thereof. In one embodiment, the non-petrochemical based component-containing compositions comprise a polymer comprising glycolic acid. As used herein, terrestrial plant matter or non-petrochemical based components include carbon atoms that have a non-petrochemical based origin. Such non-petrochemical based (or bio based or renewable) components have a ¹⁴C amount substantially higher than zero, such as about 1 parts per trillion or more, because they are derived from photosynthesis based starting material.

### DETAILED DESCRIPTION

### Definitions

Throughout this disclosure, various publications, patents, published patent applications, and the likes may be referenced by an identifying citation. The disclosures of these publications, patents and published patent applications are hereby incorporated by reference into the present disclosure in their entirety to more fully describe the state of the art to which this invention pertains.

The practice of the present technology will employ, unless otherwise indicated, conventional techniques of organic chemistry, pharmacology, immunology, molecular biology, microbiology, cell biology and recombinant DNA, which are within the skill of the art. See, e.g., Sambrook, Fritsch and Maniatis, Molecular Cloning: A Laboratory Manual, 2^{nd} edition (1989); Current Protocols in Molecular Biology (F. M. Ausubel, et al. eds., (1987)); the series Methods in Enzymology (Academic Press, Inc.): PCR 2: A Practical Approach (M. J. MacPherson, B. D. Flames and G. R. Taylor eds. (1995)), Harlow and Lane, eds. (1988) Antibodies, a Laboratory Manual, and Animal Cell Culture (R. I. Freshney, ed. (1987)).

As used in the specification and claims, the singular form “a,” “an” and “the” include plural references unless the context clearly dictates otherwise.

As used herein, the term “comprising” is intended to mean that the compounds, compositions and processes include the recited elements, but not exclude others. “Consisting essentially of” when used to define compounds, compositions and processes, shall mean excluding other elements of any essential significance to the combination. Thus, a composition consisting essentially of the elements as defined herein would not exclude trace contaminants, e.g., from the isolation and purification method. “Consisting of” shall mean excluding more than trace elements of other ingredients. Embodiments defined by each of these transition terms are within the scope of this technology.

All numerical designations, e.g., pH, temperature, time, concentration, and molecular weight, including ranges, are approximations which are varied (+) or (−) by increments of 1, 5, or 10%, e.g., by using the prefix, “about.” It is to be understood, although not always explicitly stated that all numerical designations are preceded by the term “about.” It also is to be understood, although not always explicitly stated, that the reagents described herein are merely exemplary and that equivalents of such are known in the art. As used herein, the range, “about x to y” includes about x to about y.

A “salt” is derived from a variety of organic and inorganic counter ions well known in the art and include, when the compound contains an acidic functionality, by way of example only, sodium, potassium, calcium, magnesium, ammonium, and tetraalkylammonium; and when the molecule contains a basic functionality, salts of organic or inorganic acids, such as hydrochloride, hydrobromide, tartrate, mesylate, acetate, maleate, and oxalate. Salts include acid addition salts formed with inorganic acids or organic acids. Inorganic acids suitable for forming acid addition salts include, by way of example and not limitation, hydrohalide acids (e.g., hydrochloric acid, hydrobromic acid, hydroiodic acid, etc.), sulfuric acid, nitric acid, phosphoric acid, and the like.

Organic acids suitable for forming acid addition salts include, by way of example and not limitation, acetic acid, trifluoroacetic acid, propionic acid, hexanoic acid, cyclopentanepropionic acid, glycolic acid, oxalic acid, pyruvic acid, lactic acid, malonic acid, succinic acid, malic acid, maleic acid, fumaric acid, tartaric acid, citric acid, palmitic acid, benzoic acid, 3-(4-hydroxybenzoyl) benzoic acid, cinnamic acid, mandelic acid, alkylsulfonic acids (e.g., methanesulfonic acid, ethanesulfonic acid, 1,2-ethane-disulfonic acid, 2-hydroxyethanesulfonic acid, etc.), arylsulfonic acids (e.g., benzenesulfonic acid, 4-chlorobenzenesulfonic acid, 2-naphthalenesulfonic acid, 4-toluenesulfonic acid, camphorsulfonic acid, etc.), glutamic acid, hydroxynaphthoic-acid, salicylic acid, stearic acid, muconic acid, and the like.

Salts also include salts formed when an acidic proton present in the parent compound is either replaced by a metal ion (e.g., an alkali metal ion, an alkaline earth metal ion, or an aluminum ion) or by an ammonium ion (e.g., an ammonium ion derived from an organic base, such as, ethanolamine, diethanolamine, triethanolamine, morpholine, piperidine, dimethylamine, diethylamine, triethylamine, and ammonia).

The term “accession number” and similar terms such as “protein accession number”, “UniProt ID”, “gene ID” and “gene accession number” refer to designations given to specific proteins or genes. These identifiers described a gene or protein sequence in publicly accessible databases such as the National Center for Biotechnology Information (NCBI).

The term “heterologous” as used herein refers to a material that is non-native to a cell. For example, a nucleic acid is heterologous to a cell, and so is a “heterologous nucleic acid” with respect to that cell, if at least one of the following is true: 1) the nucleic acid is not naturally found in that cell (that is, it is an “exogenous” nucleic acid); 2) the nucleic acid is naturally found in a given host cell (that is, “endogenous to”), but the nucleic acid or the RNA or protein resulting from transcription and translation of this nucleic acid is produced or present in the host cell in an unnatural (e.g., greater or lesser than naturally present) amount; 3) the nucleic acid comprises a nucleotide sequence that encodes a protein endogenous to a host cell but differs in sequence from the endogenous nucleotide sequence that encodes that same protein (having the same or substantially the same amino acid sequence), typically resulting in the protein being produced in a greater amount in the cell, or in the case of an enzyme, producing a mutant version possessing altered (e.g., higher or lower or different) activity; and/or 4) the nucleic acid comprises two or more nucleotide sequences that are not found in the same relationship to each other in the cell. As another example, a protein is heterologous to a host cell if it is produced by translation of RNA or the corresponding RNA is produced by transcription of a heterologous nucleic acid. Further, a protein is also heterologous to a host cell if it is a mutated version of an endogenous protein, and the mutation was introduced by genetic engineering.

The term “homologous”, as well as variations thereof, such as “homology”, refers to the similarity of a nucleic acid or amino acid sequence, typically in the context of a coding sequence for a gene or the amino acid sequence of a protein. Homology searches can be employed using a known amino acid or coding sequence (the “reference sequence”) for a useful protein to identify homologous coding sequences or proteins that have similar sequences and thus are likely to perform the same useful function as the protein defined by the reference sequence. As will be appreciated by those of skill in the art, a protein having homology to a reference protein is determined, for example and without limitation, by a BLAST (https://blast.ncbi.nlm.nih.gov) search. A protein with high percent homology is highly likely to carry out the identical biochemical reaction as the reference protein. In some cases, two enzymes having greater than 60% homology will carry out identical biochemical reactions, and the higher the homology, i.e., 60%, 70%, 80%, 90% or greater than 95% homology, the more likely the two proteins have the same or similar function. A protein with at least 60% homology to its reference protein is defined as substantially homologous. Any protein substantially homologous to a reference sequence can be used in a host cell according to the present disclosure.

Generally, homologous proteins share substantial sequence homology. Sets of homologous proteins generally possess one or more specific amino acids that are conserved across all members of the consensus sequence protein class. The percent sequence homology of a protein relative to a consensus sequence is determined by aligning the protein sequence against the consensus sequence. Practitioners in the art will recognized that various sequence alignment algorithms are suitable for aligning a protein with a consensus sequence. See, for example, Needleman, S B, et al., “A general method applicable to the search for similarities in the amino acid sequence of two proteins.” Journal of Molecular Biology 48 (3): 443-53 (1970). Following alignment of the protein sequence relative to the consensus sequence, the percentage of positions where the protein possesses an amino acid described by the same position in the consensus sequence determines the percent sequence homology. When a degenerate amino acid is present (i.e., B, Z, X, J or “+”) in a consensus sequence, any of the amino acids described by the degenerate amino acid may be present in the protein at the aligned position for the protein to be homologous to the consensus sequence at the aligned position. When it is not possible to distinguish between two closely related amino acids, the following one-letter symbol is used—“B” refers to aspartic acid or asparagine; “Z” refers to glutamine or glutamic acid; “J” refers to leucine or isoleucine; and “X” or “+” refers to any amino acid.

A dash (−) in a consensus sequence indicates that there is no amino acid at the specified position. A plus (+) in a consensus sequence indicates any amino acid may be present at the specified position. Thus, a plus in a consensus sequence herein indicates a position at which the amino acid is generally non-conserved; a homologous enzyme sequence, when aligned with the consensus sequence, can have any amino acid at the indicated “+” position.

In addition to identification of useful enzymes by percent sequence homology with a given consensus sequence, enzymes useful in the compositions and methods provided herein can also be identified by the occurrence of highly conserved amino acid residues in the query protein sequence relative to a consensus sequence. For each consensus sequence provided herein, a number of highly conserved amino acid residues are described. Enzymes useful in the compositions and methods provided herein include those that comprise a substantial number, and sometimes all, of the highly conserved amino acids at positions aligning with the indicated residues in the consensus sequence. Those skilled in the art will recognize that, as with percent homology, the presence or absence of these highly conserved amino acids in a query protein sequence can be determined following alignment of the query protein sequence relative to a given consensus sequence and comparing the amino acid found in the query protein sequence that aligns with each highly conserved amino acid specified in the consensus sequence.

Proteins that share a specific function are not always defined or limited by percent sequence homology. In some cases, a protein with low percent sequence homology with a reference protein is able to carry out the identical biochemical reaction as the reference protein. Such proteins may share three-dimensional structure which enables shared specific functionality, but not necessarily sequence similarity. Such proteins may share an insufficient amount of sequence similarity to indicate that they are homologous via evolution from a common ancestor and would not be identified by a BLAST search or other sequence-based searches. Thus, in some embodiments of the present disclosure, homologous proteins comprise proteins that lack substantial sequence similarity but share substantial functional similarity and/or substantial structural similarity.

As used herein, the term “express”, when used in connection with a nucleic acid encoding an enzyme or an enzyme itself in a cell, means that the enzyme, which may be an endogenous or exogenous (heterologous) enzyme, is produced in the cell. The term “overexpress”, in these contexts, means that the enzyme is produced at a higher level, i.e., enzyme levels are increased, as compared to the wild-type, in the case of an endogenous enzyme. Those skilled in the art appreciate that overexpression of an enzyme can be achieved by increasing the strength or changing the type of the promoter used to drive expression of a coding sequence, increasing the strength of the ribosome binding site or Kozak sequence, increasing the stability of the mRNA transcript, altering the codon usage, increasing the stability of the enzyme, and the like.

In the present disclosure, the term “genetic disruption” refers to several ways of altering genomic, chromosomal or plasmid-based gene expression. Non-limiting examples of genetic disruptions include CRISPR, RNAi, nucleic acid deletions, nucleic acid insertions, nucleic acid substitutions, nucleic acid mutations, knockouts, premature stop codons and transcriptional promoter modifications. In the present disclosure, “genetic disruption” is used interchangeably with “genetic modification”, “genetic mutation” and “genetic alteration.” Genetic disruptions give rise to altered gene expression and or altered protein activity. Altered gene expression encompasses decreased, eliminated and increased gene expression levels. In some examples, gene expression results in protein expression, in which case the term “gene expression” is synonymous with “protein expression”.

The terms “recombinant host cell”, “recombinant host microorganism”, and “strain” are used interchangeably herein to refer to a living cell that can be (or has been) transformed via insertion of an expression vector. A host cell or microorganism as described herein may be a prokaryotic cell (e.g., a microorganism of the kingdom Eubacteria) or a eukaryotic cell. As will be appreciated by one of skill in the art, a prokaryotic cell lacks a membrane-bound nucleus, while a eukaryotic cell has a membrane-bound nucleus.

The terms “ferment”, “fermentative”, and “fermentation” are used herein to describe culturing microbes under conditions to produce useful chemicals, including but not limited to conditions under which microbial growth, be it aerobic or anaerobic, occurs.

The terms “isolated” or “pure” refer to material that is substantially, e.g., greater than 50% or greater than 75%, or essentially, e.g., greater than 90%, 95%, 98% or 99%, free of components that normally accompany it in its native state, e.g., the state in which it is naturally found or the state in which it exists when it is first produced. Additionally, any reference to a “purified” material is intended to refer to an isolated or pure material.

The redox cofactor nicotinamide adenine dinucleotide, NAD, comes in 2 forms—phosphorylated and un-phosphorylated. The term “NAD(P)” refers to both phosphorylated (NADP) and un-phosphorylated (NAD) forms, and encompasses oxidized versions (NAD⁺ and NADP⁺) and reduced versions (NADH and NADPH) of both forms. The term “NAD(P)⁺” refers to the oxidized versions of phosphorylated and un-phosphorylated NAD, i.e., NAD⁺ and NADP⁺. Similarly, the term “NAD(P)H” refers to the reduced versions of phosphorylated and un-phosphorylated NAD, i.e., NADH and NADPH. When NAD(P)H is used to describe the redox cofactor in an enzyme catalyzed reaction, it indicates that NADH and/or NADPH is used. Similarly, when NAD(P)⁺ is the notation used, it indicates that NAD⁺ and/or NADP⁺ is used. Those skilled in the art will also appreciate that while many proteins may only bind either a phosphorylated or un-phosphorylated cofactor, there are redox cofactor promiscuous proteins, natural or engineered, that are indiscriminate; in these cases, the protein may use either NADH and/or NADPH. In some embodiments, enzymes that preferentially utilize either NAD(P) or NAD may carry out the same catalytic reaction when bound to either form.

The redox cofactor pyrroloquinoline quinone (PQQ), also called methoxatin, comes in reduced and oxidized forms. The term “PQQ” refers to both reduced and oxidized forms of PQQ.

Various values for temperatures, titers, yields, oxygen transfer rate (OTR), and pH are recited in the description and in the claims. It should be understood that these values are not intended to convey that an exact temperature is or was critical to function of the invention, unless that is explicitly stated. It should be understood that numerical ranges recited include the recited minimum value and the recited maximum value.

### Methods and Organisms

In one aspect, provided herein is a method for producing glycolic acid or a glycolate salt comprising culturing an organism that is a wild-type Pichia kudriavzevii or a wild-type Corynebacterium glutamicum, in a fermentation broth, for a sufficient period of time to produce glycolic acid, wherein the fermentation broth comprises ethylene glycol.

In one embodiment, the organism is wild-type Pichia kudriavzevii. In another embodiment, the organism is a wild-type Corynebacterium glutamicum.

In another embodiment, all of the glycolic acid is produced from ethylene glycol. In another embodiment, essentially all of the glycolic acid is produced from ethylene glycol. In one embodiment, the ethylene glycol is of terrestrial plant-matter or non-petrochemical origin.

In another embodiment, the glycolate salt produced is a calcium salt. In another embodiment, the glycolate salt produced is an ammonium salt. In another embodiment, the glycolate salt produced is a sodium salt.

In one embodiment, the culturing or fermentation is performed at a pH of about 2 to about 6. In another embodiment, the culturing is performed at a pH of about 2 to about 3. In one embodiment, the culturing is performed at a pH of about 2.9 or lower (i.e., at more than 1 pH unit below the glycolic acid pKa of about 3.87). In another embodiment, the culturing is performed at a pH of about 2.4 or lower (i.e., less than 1.5 pH units below the glycolic acid pKa.

Without being bound by theory, a low pH process can increase the rate of glycolate anion export from the cell. Additionally, a low pH process requires less base during the fermentation, lowering production cost. This also has the additional benefit of simplifying the purification process.

In another embodiment, the culturing is performed at a volumetric productivity of greater than 0.1 g/l/hour. In another embodiment, the culturing is performed such that the organism does not increase in dry cell weight by more than 20% during production phase. In another embodiment, the culturing is performed at an oxygen transfer rate (OTR) that is greater than 10 mmol/l/hr. In another embodiment, the culturing is performed at a temperature of between about 25° C. and about 45° C.

In another embodiment, the culturing further comprises calcium carbonate supplementation.

In another embodiment, at least 8 g/l glycolate is produced in the fermentation broth. In another embodiment, a yield of at least 0.4 g-glycolate/g-ethylene glycol is produced. In another embodiment, at least 10% (w/v) is provided to the organism and at a yield of at least 0.4 g-glycolate/g-ethylene glycol is produced.

Methods of making glycolic acid from other organisms are reported in Wei et al., J Ind Microbiol Biotechnol, 2009; Kataoka et al., Biosci Biotechnol Biochem, 2001; Zhang et al., J Biotechnol, 2016; Hua et al., Biosource Technol, 2018; Hua et al., Bioprocess and Biosystems Engineering, 2018; Franden et al., Metabolic Engineering, 2018; Yamada and Isobe, Ferment Technol, 2015; and Gao et al., Appl Biochem Biotechnol, 2014, and can be modified and adapted by a skilled artisan based on the disclosure provided herein to practice the present invention.

In another aspect, provided herein is a recombinant host cell that comprises a glycolic acid biosynthetic pathway, wherein the glycolic acid biosynthetic pathway comprises heterologous nucleic acids encoding a glycolaldehyde-producing enzyme and a glycolate-producing enzyme; wherein the heterologous nucleic acids are expressed in sufficient amounts to produce glycolic acid.

In one embodiment, the recombinant host cell is Pichia kudriavzevii. In another embodiment, the recombinant host cell is Corynebacterium glutamicum.

In another embodiment, the recombinant host cell is a yeast cell or a bacterial cell. In another embodiment, the recombinant host cell belongs to the genus selected from a group comprising Pichia, Issatchenkia, Candida, Corynebacterium or Escherichia. In another embodiment, the recombinant host cell is other than a recombinant host cell from a group consisting Saccharomyces cerevisiae, Pichia naganishii, Pichia anomala, Hansenula anomala, Hansenula octospora, Cryptococcus sp., Rhodotorula sp., Burkholderia sp., Gluconobacter industrius, Gluconobacter oxydans, Gluconobacter suboxydans, Gluoconobacter dioxyacetonicus, Pseudomonas putida, and Coryneform bacterium.

In another embodiment, the glycolaldehyde-producing enzyme is selected from a group comprising alcohol oxidase and alcohol dehydrogenase. In another embodiment, the alcohol dehydrogenase is NAD-dependent. In another embodiment, the alcohol dehydrogenase is PQQ-dependent. In another embodiment, the glycolate-producing enzyme is selected from a group comprising aldehyde oxidase and aldehyde dehydrogenase.

In another embodiment, the recombinant host cell further comprises a catalase. In another embodiment, the recombinant host cell further comprises a monocarboxylic acid transporter. In another embodiment, the recombinant host cell further comprises an ABC transporter, symporter, antiporter, or permease. In another embodiment, the recombinant host cell further comprises one or more heterologous nucleic acids encoding one or more ancillary proteins, wherein the one or more ancillary proteins functions in relieving oxidative stress, organic acid transport, redox cofactor recycling, redox cofactor biogenesis, or improving flux through the glycolic acid biosynthetic pathway. In another embodiment, the recombinant host cell further comprises a genetic disruption of one or more genes, wherein the one or more genes encodes a dicarboxylic acid transporter

In another aspect, provided herein is a method for producing glycolic acid or a glycolate salt comprising culturing an organism that is a recombinant host cell of the present disclosure for a sufficient period of time to produce glycolic acid, wherein the fermentation broth comprises ethylene glycol.

In one embodiment, no greater than 1 g/l oxalic acid is produced in the fermentation broth. In another embodiment, all of the glycolic acid is produced from ethylene glycol. In another embodiment, essentially all of the glycolic acid is produced from ethylene glycol. In another embodiment, the glycolate salt produced is a calcium salt.

In another embodiment, provided herein is a recombinant host cell, preferably a Pichia kudriavzevii host cell, wherein the aldehyde oxidase and/or the aldehyde dehydrogenase is an enzyme from the cytochrome P450 superfamily of heme-containing enzymes. In another embodiment, the alcohol oxidase and/or the aldehyde oxidase have limited over-oxidation of glycolic acid during fermentative cultivation, such that glyoxylic acid or oxalic acid are each produced in amounts less than about 30% g/g relative to the mass of glycolic acid produced. In another embodiment, glyoxylic acid or oxalic acid are each produced in amounts of less than about 10% g/g relative to the mass of glycolic acid produced. In another embodiment, glyoxylic acid or oxalic acid are each produced in amounts of less than about 3% g/g relative to the mass of glycolic acid produced. In another embodiment, glyoxylic acid or oxalic acid are each produced in amounts of less than about 1% g/g, relative to the mass of glycolic acid produced.

In another embodiment, the fermentation is performed to limit the over-oxidation of glycolic acid, such that glyoxylic acid or oxalic acid are produced in amounts less than about 30% g/g relative to the mass of glycolic acid produced. In another embodiment, the glyoxylic acid or oxalic acid are produced in amounts less than about 10% g/g relative to the mass of glycolic acid produced. In another embodiment, the glyoxylic acid or oxalic acid are produced in amounts less than about 3% g/g. In another embodiment, the glyoxylic acid or oxalic acid are produced in amounts or less than about 1% g/g, relative to the mass of glycolic acid produced.

In another embodiment, the culturing is performed at a volumetric productivity of greater than 0.1 g/l/hour. In another embodiment, the culturing is performed such that the organism does not increase in dry cell weight by more than 20% during production phase.

In another embodiment, the culturing is performed at an oxygen transfer rate (OTR) that is greater than 10 mmol/l/hr. In another embodiment, the culturing is performed at a temperature of between about 25° C. and about 45° C. In another embodiment, the culturing further comprises calcium carbonate supplementation. In another embodiment, at 8 g/l glycolate is produced in the fermentation broth. In another embodiment, a yield of at least 0.4 g-glycolate/g-ethylene glycol is produced. In another embodiment, at least 10% (w/v) glucose is provided to the organism and at a yield of at least 0.4 g-glycolate/g-ethylene glycol is produced.

In another embodiment, the culturing is performed at a pH of about 2 to 6. In another embodiment, the culturing is performed at a pH of about 2 to about 3. In one embodiment, the culturing is performed at a pH of about 2.9 or lower (i.e., at more than about 1 pH unit below the glycolic acid pKa of about 3.87). In another embodiment, the culturing is performed at a pH of about 2.4 or lower (i.e., less than about 1.5 pH units below the glycolic acid pKa.

In certain aspects, the glycolic acid provided herein, or a salt or derivative thereof is employed as at least one type of polymerization material to produce a glycolic acid polymer. Examples of polymerization material that is employed, comprise, for example, glycolic acid, or derivatives (such as lactides), and prepolymers and oligomers resulting from polymerizing such monomers to suitable lengths. In some embodiments, the polymerization further comprises D- or L-lactic acid or derivatives (such as lactides) and prepolymers and oligomers thereof.

Non-limiting examples of glycolic acid polymers include homopolymers of glycolic acid, heteropolymers such as hetero-block polymers, and various types of heteropolymers of glycolic acid and non-glycolic acid based (e.g., and without limitation, other hydroxy acids, such as lactic acid) polymerization material.

In some embodiments, the glycolic acid polymerization materials, or the mixture of glycolic acid polymerization material and a non-glycolic acid polymerization material, can be reacted with a suitable polymerization initiator to produce lactic acid polymers. Various Lewis acid-metal catalysts such as dioctyl stannate and the likes, and non-nucleophilic Lewis bases, such as diazabicycloundecane and the likes may be utilized as a polymerization initiator or polymerization catalyst. The polymerization is performed in solution, in a melt, or in a suspension.

### EXAMPLES

### Abbreviations

DCW: dry cell weight
HPLC: high-performance liquid chromatography
OD₆₀₀: optical density at 600 nm
Qp: specific productivity, g-product/g-DCW/hour
YPE: yeast-peptone-ethanol medium that comprises yeast extract 10 g/l, bacto peptone 20 g/l, and ethanol 2% (v/v).
BM02: medium that comprises 4.3 g/l urea, 1 g/l KH₂PO₄, 0.816 g/l K₂SO₄, 0.304 g/l MgSO₄·7H₂O, 0.124 g/l Na₂SO₄, 23.8 mg/l FeSO₄·7H₂O, 22 mg/l EDTA, 8 mg/l ZnSO₄·7H₂O, 2.3 mg/l MnCl₂, 2 mg/l myo-inositol, 0.786 mg/l/CuSO₄·5H₂O, 0.5 mg/l H₃BO₃, 0.4 mg/l calcium pantothenate, 0.4 mg/l nicotinic acid, 0.4 mg/l pyridoxal hydrochloride, 0.4 mg/l thiamine hydrochloride, 0.2 mg/l NazMoO₄, 0.2 mg/l p-aminobenzoic acid, 0.2 mg/l riboflavin, 2 μg/l biotin, and 2 μg/l folic acid.

### Example 1: Production of Glycolic Acid from Ethylene Glycol with Wild-Type Pichia kudriavzevii

This example describes the culturing and analysis of wild-type P. kudriavzevii for the production of glycolic acid from ethylene glycol. An overnight culture of P. kudriavzevii was grown at 30° C. in YPE medium. Colonies were used to inoculate replicate tubes of YPE medium and incubated at 30° C. with 80% humidity and shaking at 250 rpm for 20 hours. These replicate tubes of overnight pre-cultures were used to inoculate baffled flask replicates with BM02 medium and 5-10% (v/v) ethylene glycol. Pre-cultures were diluted for OD₆₀₀ measurements to inform appropriate dilution of pre-cultures to produce a starting culture biomass of ˜5 g-DCW/1. Baffled flask cultures were then incubated at 30° ° C. with 80% humidity and shaking at 250 rpm. Cells were cultured at 30° C. for ˜72 hours and glycolate fermentation metrics were recorded. At 24 hours, 48 hours, and 72 hours, samples were taken from the flask cultures for HPLC analysis. Samples of cultures were diluted 10× with 12 M HCl, spin-filtered and frozen for storage. Samples were analyzed by HPLC within 48 hours of harvest.

For HPLC analysis, samples were analyzed by Shimadzu LC-20 instrumentation using a Bio-Rad Aminex 87H column (300×7.8 mm) kept at 50° ° C. with an isocratic gradient of 0.6 ml/min for 22 min. The mobile phase was 7.5 mM sulfuric acid in water. The detection used was Refractive index and UV at 210 nm wavelength.

The wild-type P. kudriavzevii produced the following fermentation metrics at 24, 48, and 72-hour timepoints: (1) At 24 hours, titer was 9.23-11.81 g/l glycolate, volumetric productivity was 0.38-0.5 g/l/hour, Qp was 0.09-0.13 g/g-DCW-hour, 9-25 g/l of ethylene glycol was consumed, and yield was 0.4-0.7 g-glycolate/g-ethylene glycol. (2) At 48 hours, titer was 11.26-17.84 g/l glycolate, volumetric productivity was 0.24-0.37 g/l/hour, Qp was 0.06-0.11 g/g-DCW-hour, and yield was 0.9-1.0 g-glycolate/g-ethylene glycol. (3) At 72 hours, titer was 10.71-16.83 g/l glycolate, volumetric productivity was 0.15-0.23 g/l/hour, Qp was 0.03-0.07 g/g-DCW-hour, and yield was 0.4-1.3 g-glycolate/g-ethylene glycol. At 24, 48 and 72 hours, culture pH was pH 3.

### Example 2: Production of Glycolic Acid from Ethylene Glycol with Wild-Type Pichia kudriavzevii and 100 mM Methylmalonate pH 5 Supplementation

This example describes the culturing and analysis of wild-type P. kudriavzevii for the production of glycolic acid from ethylene glycol with 100 mM methylmalonate pH 5 (from a 1M methylmalonate stock that was titrated with NaOH pellets to a final pH of 5) supplementation for pH control. The culturing and fermentation of wild-type P. kudriavzevii in this example were carried out as described above in Example 1 with the following exception—replicate tubes of overnight pre-cultures were used to inoculate baffled flask replicates with BM02 medium and 5-10% (v/v) ethylene glycol and 100 mM methylmalonate pH 5. The samples collected at 24, 48 and 72 hours were treated and analyzed by HPLC as described above in Example 1 as well.

The wild-type P. kudriavzevii produced the following fermentation metrics at 24, 48, and 72-hour timepoints: (1) At 24 hours, titer was 9.32-16.11 g/l glycolate, volumetric productivity was 0.39-0.67 g/l/hour, Qp was 0.09-0.17 g/g-DCW-hour, 9-25 g/l of ethylene glycol was consumed, and yield was 0.5-0.7 g-glycolate/g-ethylene glycol. (2) At 48 hours, titer was 12.94-19.86 g/l glycolate, volumetric productivity was 0.27-0.41 g/l/hour, Qp was 0.07-0.13 g/g-DCW-hour, and yield was 0.7-1.1 g-glycolate/g-ethylene glycol. (3) At 72 hours, titer was 12.53-20.74 g/l glycolate, volumetric productivity was 0.17-0.29 g/l/hour, Qp was 0.05-0.09 g/g-DCW-hour, and yield was 1-1.2 g-glycolate/g-ethylene glycol. At 24, 48 and 72 hours, culture pH was pH 4.

### Example 3: Production of Glycolic Acid from Ethylene Glycol with Wild-Type Pichia kudriavzevii and 40 g/l Calcium Carbonate pH 6 Supplementation

This example describes the culturing and analysis of wild-type P. kudriavzevii for the production of glycolic acid from ethylene glycol with 40 g/l CaCO₃ supplementation. The culturing and fermentation of wild-type P. kudriavzevii in this example were carried out as described above in Example 1 with the following exception—replicate tubes of overnight pre-cultures were used to inoculate baffled flask replicates with BM02 medium and 5-10% (v/v) ethylene glycol and 40 g/l CaCO₃ at pH 6. The samples collected at 24, 48 and 72 hours were treated and analyzed by HPLC as described above in Example 1 as well. A significant amount of white precipitate was observed in the shake flasks. A representative sample of the culture with the white precipitate was acidified with HCl and analyzed by HPLC as described above in Example 1—HPLC results confirmed that the white precipitate is calcium glycolate.

The wild-type P. kudriavzevii produced the following fermentation metrics at 24, 48, and 72-hour timepoints: (1) At 24 hours, titer was 13.87-18.5 g/l glycolate, volumetric productivity was 0.57-0.77 g/l/hour, Qp was 0.16-0.22 g/g-DCW-hour, 9-25 g/l of ethylene glycol was consumed, and yield was 0.5-1.6 g-glycolate/g-ethylene glycol. (2) At 48 hours, titer was 22.93-27.12 g/l glycolate, volumetric productivity was 0.48-0.56 g/l/hour, Qp was 0.17-0.27 g/g-DCW-hour, and yield was 0.7-1.1 g-glycolate/g-ethylene glycol. (3) At 72 hours, titer was 21.63-28.57 g/l glycolate, volumetric productivity was 0.3-0.39 g/l/hour, Qp was 0.16-0.24 g/g-DCW-hour, and yield was 0.7-1.1 g-glycolate/g-ethylene glycol. At 24, 48 and 72 hours, culture pH was around pH 5-6.

### Example 4: Production of Glycolic Acid from Ethylene Glycol with Wild-Type Pichia kudriavzevii, 40 g/l Calcium Carbonate, and 10% (w/v) Glucose Supplementation

This example describes the culturing and analysis of wild-type P. kudriavzevii for the production of glycolic acid from ethylene glycol with 10% (w/v) glucose supplementation. The culturing and fermentation of wild-type P. kudriavzevii in this example were carried out as described above in Example 1 with the following exceptions—(1) replicate tubes of overnight pre-cultures were used to inoculate baffled flask replicates with BM02 medium and 10% (v/v) ethylene glycol, 40 g/l CaCO₃, and 10% (w/v) glucose; and (2) the starting biomass was ˜10, ˜20, ˜30, and ˜40 g-DCW/1. The samples collected at 24, 48 and 72 hours were treated and analyzed by HPLC as described above in Example 1 as well. A significant amount of precipitate was observed in the shake flasks. A representative sample of the culture with the white precipitate was acidified with HCl and analyzed by HPLC as described above in Example 1—HPLC results confirmed that the white precipitate is calcium glycolate.

The wild-type P. kudriavzevii with the starting biomass of ˜10 g-DCW/l produced the following fermentation metrics at 24, and 48-hour timepoints: (1) At 24 hours, titer was 22.52-30.27 g/l glycolate, volumetric productivity was 0.398-1.32 g/l/hour, Qp was 0.03 g/g-DCW-hour, and yield was 1 g-glycolate/g-ethylene glycol. (2) At 48 hours, titer was 41.56 g/l glycolate, volumetric productivity was 0.88 g/l/hour, Qp was 0.04 g/g-DCW-hour, and yield was ˜1 g-glycolate/g-ethylene glycol.

The wild-type P. kudriavzevii with the starting biomass of ˜20 g-DCW/l produced the following fermentation metrics at 24, and 48-hour timepoints: (1) At 24 hours, titer was 22.33-23.25 g/l glycolate, volumetric productivity was 0.97-1.01 g/l/hour, Qp was 0.03 g/g-DCW-hour, and yield was 1 g-glycolate/g-ethylene glycol. (2) At 48 hours, titer was 40.61 g/l glycolate, volumetric productivity was 0.86 g/l/hour, Qp was 0.04 g/g-DCW-hour, and yield was ˜1 g-glycolate/g-ethylene glycol.

The wild-type P. kudriavzevii with the starting biomass of ˜30 g-DCW/l produced the following fermentation metrics at 24, and 48-hour timepoints: (1) At 24 hours, titer was 20.53 g/l glycolate, volumetric productivity was 0.89 g/l/hour, Qp was 0.03 g/g-DCW-hour, and yield was 1 g-glycolate/g-ethylene glycol. (2) At 48 hours, titer was 41.56 g/l glycolate, volumetric productivity was 1.06 g/l/hour, Qp was 0.04 g/g-DCW-hour, and yield was ˜1 g-glycolate/g-ethylene glycol.

The wild-type P. kudriavzevii with the starting biomass of ˜40 g-DCW/l produced the following fermentation metrics at 24, and 48-hour timepoints: (1) At 24 hours, titer was 20.47-26.16 g/l glycolate, volumetric productivity was 0.89 g/l/hour, Qp was 0.03 g/g-DCW-hour, and yield was 1 g-glycolate/g-ethylene glycol. (2) At 48 hours, titer was 51.32 g/l glycolate, volumetric productivity was 1.09 g/l/hour, Qp was 0.03 g/g-DCW-hour, and yield was ˜1 g-glycolate/g-ethylene glycol.

### Example 5: Production of Glycolic Acid from Ethylene Glycol with Wild-Type Corynebacterium glutamicum

This example describes the culturing and analysis of wild-type C. glutamicum for the production of glycolic acid from ethylene glycol. An overnight culture of C. glutamicum was grown at 37° C. in commercially available brain heart infusion medium (BHI) with 2% ethanol. Colonies were used to inoculate replicate tubes of BHI medium and incubated at 30° ° C. with 80% humidity and shaking at 250 rpm for 20 hours. These replicate tubes of overnight pre-cultures were used to inoculate baffled flask replicates with CGXII medium and 5-10% (v/v) ethylene glycol. Pre-cultures were diluted for OD₆₀₀ measurements to inform appropriate dilution of pre-cultures to produce a starting culture biomass of 4.5-5 g-DCW/1. Baffled flask cultures were then incubated at 37° C. with 80% humidity and shaking at 250 rpm. Cells were cultured at 37° C. for ˜70 hours and glycolate fermentation metrics were recorded. At 24 hours, 48 hours, and 70 hours, samples were taken from the flask cultures for HPLC analysis. Samples of cultures were diluted 10× with 12 M HCl, spin-filtered and frozen for storage. Samples were analyzed by HPLC within 48 hours of harvest.

For HPLC analysis, samples were analyzed by Shimadzu LC-20 instrumentation using a Bio-Rad Aminex 87H column (300×7.8 mm) kept at 50° C. with an isocratic gradient of 0.6 ml/min for 22 min. The mobile phase was 7.5 mM sulfuric acid in water. The detection used was Refractive index and UV at 210 nm wavelength.

The wild-type C. glutamicum produced the following fermentation metrics at 24, 48 and 70-hour timepoints: (1) At 24 hours, titer was 2.77-3.03 g/l glycolate, volumetric productivity was 0.115-0.2126 g/l/hour, and Qp was 0.029-0.032 g/g-DCW-hour. (2) At 48 hours, titer was 3.16-4.0 g/l glycolate, volumetric productivity was 0.066-0.085 g/l/hour, and Qp was 0.015-0.018 g/g-DCW-hour. (3) At 70 hours, titer was 2.26-3.77 g/l glycolate, volumetric productivity was 0.032-0.054 g/l/hour, Qp was 0.006-0.010 g/g-DCW-hour.

### Example 6: Production of Glycolic Acid from Ethylene Glycol with Wild-Type Corynebacterium glutamicum and 100 mM Methylmalonate pH 5 Supplementation

The culturing and fermentation of wild-type C. glutamicum in this example were carried out as described above in Example 5 with the following exception—replicate tubes of overnight pre-cultures were used to inoculate baffled flask replicates with 100 mM methylmalonate pH 5 (from a 1M methylmalonate stock that was titrated with NaOH pellets to a final pH of 5). The samples collected at 24, 48 and 70 hours were treated and analyzed by HPLC as described above in Example 5 as well.

The wild-type C. glutamicum produced the following fermentation metrics at 24, 48 and 70-hour timepoints: (1) At 24 hours, titer was 2.54-2.86 g/l glycolate, volumetric productivity was 0.106-0.119 g/l/hour, and Qp was 0.026-0.029 g/g-DCW-hour. (2) At 48 hours, titer was 2.99-3.52 g/l glycolate, volumetric productivity was 0.062-0.073 g/l/hour, and Qp was 0.013-0.0159 g/g-DCW-hour. (3) At 70 hours, titer was 2.21-2.80 g/l glycolate, volumetric productivity was 0.032-0.04 g/l/hour, Qp was 0.006-0.008 g/g-DCW-hour.

### Example 7: Production of Glycolic Acid from Ethylene Glycol with Wild-Type Corynebacterium glutamicum and 40 g/l Calcium Carbonate Supplementation

The culturing and fermentation of wild-type C. glutamicum in this example were carried out as described above in Example 5 with the following exception—replicate tubes of overnight pre-cultures were used to inoculate baffled flask replicates with 40 g/l CaCO₃. The samples collected at 24, 48 and 70 hours were treated and analyzed by HPLC as described above in Example 5 as well.

The wild-type C. glutamicum produced the following fermentation metrics at 24, 48 and 70-hour timepoints: (1) At 24 hours, titer was 2.68-3.33 g/l glycolate, volumetric productivity was 0.112-0.139 g/l/hour, and Qp was 0.023-0.27 g/g-DCW-hour. (2) At 48 hours, titer was 3.14-4.67 g/l glycolate, volumetric productivity was 0.066-0.09 g/l/hour, and Qp was 0.013-0.017 g/g-DCW-hour. (3) At 70 hours, titer was 2.43-3.99 g/l glycolate, volumetric productivity was 0.035-0.057 g/l/hour, Qp was 0.000-0.010 g/g-DCW-hour.

### Example 8: Construction of Recombinant P. kudriavzevii Comprising a Heterologous Alcohol Dehydrogenase

Example 8 describes the construction of recombinant P. kudriavzevii host cells of the present disclosure that each comprised heterologous nucleic acids encoding an alcohol dehydrogenase. Alcohol dehydrogenases were selected from the following: Escherichia coli. FucO, Pseudomonas aeruginosa AdhA, Homo sapiens ADH1B1, Homo sapiens ADH1B2, Homo sapiens ADH1C1, Homo sapiens ADH1C2, Clostridium sp. Ldr, Bacillus sp. ADH, Cronobacter sakazakii ADH, Photobacterium marinum EutG, Desulfovibrio gracilis ADH, Acetobacter pasteurianus ADH, Gammaproteobacteria bacterium ADH, Beobacteria bacterium ADH, Candidatus rokubacteria ADH, Hyalangium minutum ADH, Actinomycetospora cinnamomea ADH, Geodermatophilus amargosae ADH, Pyrobaculum calidifontis ADH, Maledivibacter halophilus ADH, Chloroflexi bacterium ADH, Adrococcus urinae ADH, Pongo abelii ADH, Frankia sp. ADH, Truepera radiovictrix ADH, Mminiimonas sp. ADH, Oscillibacter sp. ADH, Propionibacterium feurdenreichii ADH.

Fifty-eight P. kudriavzevii strains, LPK1511109-1511166, comprising different ADH homologs and sister clones were generated.

The heterologous nucleic acids used in this example were codon-optimized for yeast and were synthesized. Each gene was cloned into its own entry vector, pEV, along with an upstream transcriptional promoter and a downstream transcriptional terminator. The transcriptional promoter cloned in front (5′) of each gene was the constitutive P. kudriavzevii TDH1 promoter (pPKTDH1). The transcriptional terminator cloned behind (3′) of each gene was the S. cerevisiae TEF1 terminator (tScTEF1). Additionally, a HIS3 marker was included in the heterologous expression cassette to complement the histidine auxotrophic deficiency in the parent strain. This HIS3 marker comprised a transcriptional promoter, a HIS3 coding region, and a transcriptional terminator. The transcriptional promoter 5′ of HIS3 was the P. kudriavzevii TEF1 promoter (pPKTEF1) and the transcriptional terminator 3′ of HIS3 was the S. cerevisiae TDH3 terminator (tScTDH3).

Each gene was amplified from its respective pEV vector using primers with upstream and downstream homologous regions to neighboring genetic elements to drive correct assembly of the full-length pathway. The upstream and downstream homologous regions were 25 bp to 700 bp in length. The 5′ and 3′ ends of the expression cassette comprised regions homologous to the genomic sequences upstream and downstream of the P. kudriavzevii YAT1 locus, thereby facilitating integration of the heterologous nucleic acids encoding the glycolic acid pathway enzymes at the YAT1 locus in the P. kudriavzevii genome. Consequently, one or both copies of the PkYAT1 gene were deleted from the host genome; thus, genomic integration of the glycolic acid pathway simultaneously decreased or eliminated expression of PKYAT1.

All PCR products were purified and provided as exogenous nucleic acids to P. kudriavzevii. Transformation was carried out in a single step. Transformants were selected on CSM-His medium. Successful integration of all heterologous nucleic acids encoding the first 2 glycolic acid pathway enzymes as well as deletion of one or both copies of the genes encoding PkYAT1 were confirmed by genetic sequencing of this locus and the flanking regions.

### Example 9: Construction of Recombinant P. kudriavzevii Comprising a Heterologous Aldehyde Dehydrogenase

Recombinant P. kudriavezvii host cells of the present disclosure that each comprise heterologous nucleic acids encoding an aldehyde dehydrogenase are constructed as described above and replacing alcohol dehydrogenase with aldehyde dehydrogenase.

It should be noted that there may be alternative ways of implementing the embodiments disclosed herein. Accordingly, the present embodiments are to be considered as illustrative and not restrictive; various modifications can be made without departing from the spirit of the invention. Furthermore, the claims are not to be limited to the details given herein, and are entitled their full scope and equivalents thereof.

What is claimed is:

1. A method for producing glycolic acid or a salt thereof comprising:
culturing an organism that is a wild-type Pichia kudriavzevii in a fermentation broth, for a sufficient period of time to produce glycolic acid or the salt thereof,
wherein the fermentation broth comprises ethylene glycol.

2. The method of claim 1, wherein all or essentially all of the glycolic acid is produced from the ethylene glycol.

3. The method of claim 1, wherein the salt is a calcium, ammonium, or sodium salt.

4. The method of claim 1, wherein the culturing is performed at a volumetric productivity of greater than 0.1 g/l/hour.

5. The method of claim 1, wherein the culturing is performed such that the organism does not increase in dry cell weight by more than 20% during production phase.

6. The method of claim 1, wherein the culturing is performed at a temperature of between about 25° C. and about 45° C.

7. The method of claim 1, wherein the culturing is performed such that the fermentation pH is about 2 to about 6, or about 2 to about 3.

8. The method of claim 1, further comprising calcium carbonate supplementation.

9. The method of claim 1, further comprising producing at least 8 g/l glycolate in the fermentation broth.

10. The method of claim 1, further comprising producing a glycolic acid yield of at least 0.4 g-glycolate/g-ethylene glycol.

11. The method of claim 1, further comprising
providing at least 10% (w/v) glucose to the organism and producing a glycolic acid yield of at least 0.4 g-glycolate/g-ethylene glycol.

12. A recombinant host cell comprising a glycolic acid biosynthetic pathway; wherein the glycolic acid biosynthetic pathway comprises heterologous nucleic acids encoding a glycolaldehyde-producing enzyme and a glycolate-producing enzyme; wherein the heterologous nucleic acids are expressed in sufficient amounts to produce glycolic acid or a salt thereof.

13. The recombinant host cell of claim 12, wherein the recombinant host cell is a yeast cell or a bacterial cell.

14. The recombinant host cell of claim 12, wherein the recombinant host cell belongs to the genus selected from a group comprising Pichia, Issatchenkia, Candida, Corynebacterium or Escherichia.

15. The recombinant host cell of claim 12, wherein the recombinant host cell is Pichia kudriavzevii or Corynebacterium glutamicum.

16. The recombinant host cell of claim 12, wherein the recombinant host cell is other than a recombinant host cell from a group consisting Saccharomyces cerevisiae, Pichia naganishii, Pichia anomala, Hansenula anomala, Hansenula octospora, Cryptococcus sp., Rhodotorula sp., Burkholderia sp., Gluconobacter industrius, Gluconobacter oxydans, Gluconobacter suboxydans, Gluoconobacter dioxyacetonicus, Pseudomonas putida, and Coryneform bacterium.

17. The recombinant host cell of claim 12, wherein the glycolaldehyde-producing enzyme is selected from a group comprising alcohol oxidase and alcohol dehydrogenase.

18. The recombinant host cell of claim 17, wherein the alcohol dehydrogenase is NAD-dependent.

19. The recombinant host cell of claim 17, wherein the alcohol dehydrogenase is PQQ-dependent.

20. The recombinant host cell of claim 12, wherein the glycolate-producing enzyme is selected from a group comprising aldehyde oxidase and aldehyde dehydrogenase.

21. The recombinant host cell of claim 12, wherein the aldehyde oxidase and/or the aldehyde dehydrogenase is an enzyme from the cytochrome P450 superfamily of heme-containing enzymes.

22. The recombinant host cell of claim 12, wherein the alcohol oxidase and/or the aldehyde oxidase are selected from variants which result in limited over-oxidation of glycolic acid during fermentative cultivation, such that glyoxylic acid and oxalic acid are each produced in amounts <30% g/g, or less than 10% g/g, or less than 3% g/g, or less than 1% g/g, relative to the mass of glycolic acid produced.

23. The method of claim 1, wherein the parameters used during the cultivation are controlled to limit the over-oxidation of glycolic acid, such that glyoxylic acid and oxalic acid are each produced in amounts <30% g/g, or less than 10% g/g, or less than 3% g/g, or less than 1% g/g, relative to the mass of glycolic acid produced.

24. The recombinant host cell of claim 12, further comprising:
a catalase.

25. The recombinant host cell of claim 12, further comprising:
a monocarboxylic acid transporter.

26. The recombinant host cell of claim 12, further comprising:
an ABC transporter, symporter, antiporter, or permease.

27. The recombinant host cell of claim 12, further comprising:
one or more heterologous nucleic acids encoding one or more ancillary proteins;
wherein the one or more ancillary proteins functions in relieving oxidative stress, organic acid transport, redox cofactor recycling, redox cofactor biogenesis, or improving flux through the glycolic acid biosynthetic pathway.

28. The recombinant host cell of claim 12, further comprising a genetic disruption of one or more genes, wherein the one or more genes encodes a dicarboxylic acid transporter.

29. A method for producing glycolic acid or a salt thereof comprising:
culturing the recombinant host cell of claim 12 in a fermentation broth for a sufficient period of time to produce glycolic acid, wherein the fermentation broth comprises ethylene glycol.

30. The method of claim 29, wherein the salt is a calcium, ammonium, or sodium salt.

31. The method of claim 29, wherein the culturing is performed at a volumetric productivity of greater than 0.1 g/l/hour.

32. The method of claim 29, wherein the culturing is performed such that the organism does not increase in dry cell weight by more than 20% during production phase.

33. The method of claim 29, wherein the culturing is performed at a temperature of between about 25° C. and about 45° C.

34. The method of claim 29, wherein the culturing is performed such that the fermentation pH is about 2 to 6, or about 2 to about 3.

35. The method of claim 29, further comprising calcium carbonate supplementation.

36. The method of claim 29, further comprising producing at least 8 g/l glycolate in the fermentation broth.

37. The method of claim 29, further comprising
producing a glycolic acid yield of at least 0.4 g-glycolate/g-ethylene glycol.

38. The method of claim 29, further comprising
providing at least 10% (w/v) glucose to the organism and producing a glycolic acid yield of at least 0.4 g-glycolate/g-ethylene glycol.

39. A method for producing a glycolic acid polymer, comprising:
isolating the glycolic acid or salt thereof of claim 1 or 29;
optionally converting the glycolic acid or salt thereof to a glycolic acid derivative; and
producing a glycolic acid polymer using the isolated glycolic acid, the salt thereof, or the glycolic acid derivative.

40. A glycolic acid or glycolate salt containing carbon that has an isotopic distribution that is within measurement error of the isotopic distribution contained in terrestrial plant matter, indicating that the carbon was sourced from a sustainable source rather than petroleum.
